SEQUENCE LISTING (56 sequences)

SEQ ID NO: 1 - DNA, 201 bp, Homo sapiens
gttacagtgttactattctaatcactcagactagaacactcagagctatctatgagtcttcctctcactcaaactgcatatataatctataaacaggtccygttgacctcatcttctaaaaatctcttacatgctcctttttatatcattggataatagtttagtgtggtggttaatatcatgcacactggagccaaatct

SEQ ID NO: 2 - DNA, 201 bp, Homo sapiens
ttcaataatttttttctagcagaatcccacaactgaatattgtcaagcagtttaaactgcattcgtgtttgttaaaactttaaaagggaaacttaaaactraagtatgttgtttttctgattttaatattgtgctttctagagatgaatccttttactgtttgaggcactataggaattatgtatttaaatgtatgtattt

SEQ ID NO: 3 - DNA, 201 bp, Homo sapiens
attaggggttgttcctgggtgcaaattaatatactgtcttttacatttccggaggttgttgacctggccttgaagatcagccaacagtttagaaaataagytgtgcaaattgcattgctacacctcaaataatttaaagcctgacagttctgtacagaataagagtcacataagatgtgatttgttaaagattatagaaat

SEQ ID NO: 4 - DNA, 201 bp, Homo sapiens
taatgcatgaggtctacttcttcctcagtctgaaaatatgaatattccaaaatggtactcaaggcagaggctatatatcaaaattaaaattctgtgatacyatgtttttagggaagaagaaaactgtagcattagaaatgtagacgcaaaaggtcagaacaaagtccagtatgtcaagtccctttataatgccatctgaat

SEQ ID NO: 5 - DNA, 201 bp, Homo sapiens
tagaaatttcatttaattatttaaaaaaccaacagtgtttgcttacaaaaggcatagggctgaaattgtaaattatttttaaatatgaattgtgtgagaaycaaaacaaaggaattacctctgaaacttcatctccaaaggcttcccttgtgtttagatctggaatctcccaaggaattgttttaggtctcactgtgatga

SEQ ID NO: 6 - DNA, 201 bp, Homo sapiens
aaatgagaaacctagaatttttgttttggacacactttgatcatatctaatgtgttctacttcctctaattatttttatgttattaggatttttaaaaaayctcttagattaaaatttctcagtgtatctttgtttgcgtgtctttttttctctctccacttatgtaattgaagcatttctgacatatcctcagaacacac

SEQ ID NO: 7 - DNA, 201 bp, Homo sapiens
ctctttcaaatggatccaacagcacatgagtcactggcagcatgttcagctcctttacctcaggaattacccagttcctcattcttgccacccttcagccrtgaagtagtaatatgaaaaatacatgagtactttttgaggatacctcggacctctttccttcagtaccagagactaattgcctcataatggtaacattga

SEQ ID NO: 8 - DNA, 201 bp, Homo sapiens
gcatagtactcagactcattgaaagtttatagaatttggcctgtgtggaaaactctgtgctccaagtacaacaactaactgaattctctaatttaaacaastttgaagggaagtgaaaggttataaaatgatacagactcataccgcagaatcaccttaaaatgcagctgttggagaaaaaaaaaaaggaagctttatgct

SEQ ID NO: 9 - DNA, 201 bp, Homo sapiens
ttgtttcctagtagtagacatttaaccaacttctaacttcctacctcagtagataatactgcagtgactgtcctcacttatgtcctcttgtggatttgtgycagaatttcacccagatacatctgcaagagttgggttgctgggacatttgtatatttggctttccagaatgtctgtgtaagtccccatgcccacagccat

SEQ ID NO: 10 - DNA, 201 bp, Homo sapiens
ctccctcaaaaatggcatcattagacatcaagctaaacactcataaaaatcatctaagcctctgagaagtaactctagctctagtctttgagttactttgrtgttttgtttatttgtttgttgtttttgtttttgctggatgctaaagagcagactacatttcccaaaatatttcaataattatacatcctagtaggtttg

SEQ ID NO: 11 - DNA, 201 bp, Homo sapiens
gctgagtcctgctccttccaaaacacttatggacaactatgaggtaacaccttaccttacagatttagcaattttcatttgactttactctgttaacatcwctggccagggcataatctccttcccttttccttaactctgaagaagaaaaccagtcattgcccaaggcctgggagggtgacccaggccaagttggcagcc

SEQ ID NO: 12 - DNA, 201 bp, Homo sapiens
cacaagactagattgcaggaagatgtgaaagcatttggctaattctgacatttggctaataacatcctcacagtatgtcccaagtatcttctgcccttccyaccacggtgagcagtcatgacagtgagggtggcataggtgggatgtgtggctggcagaagccagtgggcaagagttgtccacagaatagtccatgagctt

SEQ ID NO: 13 - DNA, 201 bp, Homo sapiens
ttttgaggtttctatactctgcaggcataacccagactttttcaggatcagcggtctttcttttcagcagaacgaagccctgctgctgctcatttacctayctgcgtcttctgaagtagctgaatgtggcacagacgttcctggtcaccactatggaatgcagctgcacaaactggactttgtacattttaacaggaaaaa

SEQ ID NO: 14 - DNA, 201 bp, Homo sapiens
aacatatgcacacagcgtgtgggccaaggacgattttggtccctccaccngcagaatgtaattcctttccccaaatcacgcccatagctcacagttttggycctttcctgattgttttgttactaagcctttaaatagctcatctgacttctcctctgtcaatcatctattcaggaggcttggtaccaagttttcatagat

SEQ ID NO: 15 - DNA, 30 bp, Artificial Sequence
  primer
acgttggatgtaaaaaggagcatgtaagag

SEQ ID NO: 16 - DNA, 30 bp, Artificial Sequence
  primer
acgttggatgcttcctctcactcaaactgc

SEQ ID NO: 17 - DNA, 23 bp, Artificial Sequence
  primer
gatttttagaagatgaggtcaac

SEQ ID NO: 18 - DNA, 30 bp, Artificial Sequence
  primer
acgttggatgttaaactgcattcgtgtttg

SEQ ID NO: 19 - DNA, 31 bp, Artificial Sequence
  primer
acgttggatgggattcatctctagaaagcac

SEQ ID NO: 20 - DNA, 24 bp, Artificial Sequence
  primer
actttaaaagggaaacttaaaact

SEQ ID NO: 21 - DNA, 30 bp, Artificial Sequence
  primer
acgttggatgtctgtacagaactgtcaggc

SEQ ID NO: 22 - DNA, 30 bp, Artificial Sequence
  primer
acgttggatgccttgaagatcagccaacag

SEQ ID NO: 23 - DNA, 17 bp, Artificial Sequence
  primer
gcaatgcaatttgcaca

SEQ ID NO: 24 - DNA, 30 bp, Artificial Sequence
  primer
acgttggatggctacagttttcttcttccc

SEQ ID NO: 25 - DNA, 30 bp, Artificial Sequence
  primer
acgttggatgaatggtactcaaggcagagg

SEQ ID NO: 26 - DNA, 20 bp, Artificial Sequence
  primer
ttcttcttccctaaaaacat

SEQ ID NO: 27 - DNA, 30 bp, Artificial Sequence
  primer
acgttggatgtggagatgaagtttcagagg

SEQ ID NO: 28 - DNA, 30 bp, Artificial Sequence
  primer
acgttggatggcttacaaaaggcatagggc

SEQ ID NO: 29 - DNA, 20 bp, Artificial Sequence
  primer
gaggtaattcctttgttttg

SEQ ID NO: 30 - DNA, 31 bp, Artificial Sequence
  primer
acgttggatgcgcaaacaaagatacactgag

SEQ ID NO: 31 - DNA, 30 bp, Artificial Sequence
  primer
acgttggatgctaatgtgttctacttcctc

SEQ ID NO: 32 - DNA, 24 bp, Artificial Sequence
  primer
cactgagaaattttaatctaagag

SEQ ID NO: 33 - DNA, 30 bp, Artificial Sequence
  primer
acgttggatgagttcctcattcttgccacc

SEQ ID NO: 34 - DNA, 30 bp, Artificial Sequence
  primer
acgttggatgtactgaaggaaagaggtccg

SEQ ID NO: 35 - DNA, 17 bp, Artificial Sequence
  primer
cttgccacccttcagcc

SEQ ID NO: 36 - DNA, 30 bp, Artificial Sequence
  primer
acgttggatgctgcggtatgagtctgtatc

SEQ ID NO: 37 - DNA, 30 bp, Artificial Sequence
  primer
acgttggatgctctgtgctccaagtacaac

SEQ ID NO: 38 - DNA, 21 bp, Artificial Sequence
  primer
aacctttcacttcccttcaaa

SEQ ID NO: 39 - DNA, 30 bp, Artificial Sequence
  primer
acgttggatgaaatgtcccagcaacccaac

SEQ ID NO: 40 - DNA, 30 bp, Artificial Sequence
  primer
acgttggatgctgtcctcacttatgtcctc

SEQ ID NO: 41 - DNA, 19 bp, Artificial Sequence
  primer
gtatctgggtgaaattctg

SEQ ID NO: 42 - DNA, 30 bp, Artificial Sequence
  primer
acgttggatgctctgagaagtaactctagc

SEQ ID NO: 43 - DNA, 30 bp, Artificial Sequence
  primer
acgttggatgtctgctctttagcatccagc

SEQ ID NO: 44 - DNA, 22 bp, Artificial Sequence
  primer
ctctagtctttgagttactttg

SEQ ID NO: 45 - DNA, 30 bp, Artificial Sequence
  primer
acgttggatgggtaacaccttaccttacag

SEQ ID NO: 46 - DNA, 30 bp, Artificial Sequence
  primer
acgttggatgaagggaaggagattatgccc

SEQ ID NO: 47 - DNA, 23 bp, Artificial Sequence
  primer
tttgactttactctgttaacatc

SEQ ID NO: 48 - DNA, 30 bp, Artificial Sequence
  primer
acgttggatgacatcctcacagtatgtccc

SEQ ID NO: 49 - DNA, 30 bp, Artificial Sequence
  primer
acgttggatgtcactgtcatgactgctcac

SEQ ID NO: 50 - DNA, 20 bp, Artificial Sequence
  primer
caagtatcttctgcccttcc

SEQ ID NO: 51 - DNA, 30 bp, Artificial Sequence
  primer
acgttggatggccacattcagctacttcag

SEQ ID NO: 52 - DNA, 30 bp, Artificial Sequence
  primer
acgttggatggcggtctttcttttcagcag

SEQ ID NO: 53 - DNA, 17 bp, Artificial Sequence
  primer
tacttcagaagacgcag

SEQ ID NO: 54 - DNA, 30 bp, Artificial Sequence
  primer
acgttggatgagaggagaagtcagatgagc

SEQ ID NO: 55 - DNA, 30 bp, Artificial Sequence
  primer
acgttggatgaaatcacgcccatagctcac

SEQ ID NO: 56 - DNA, 20 bp, Artificial Sequence
  primer
aacaaaacaatcaggaaagg

(19) United States
(12) Patent Application Publication
(10) Pub. No.: US 2010/0203503 A1
(43) Pub. Date: Aug. 12, 2010

(54) GENETIC POLYMORPHISMS ASSOCIATED WITH MYOCARDIAL INFARCTION AND USES THEREOF

(75) Inventors: Byung-Chul Kim, Hwaseong-si (KR); Kyung-Hee Park, Seoul (KR); Tae-Jin Ahn, Seoul (KR); Kyu-Sang Lee, Suwon-si (KR); Jae-Heup Kim, Seoul (KR); Ki-Eun Kim, Seoul (KR); Yeon-Su Lee, Goyang-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/993,549

(22) PCT Filed: May 17, 2006
(86) PCT No.: PCT/KR06/01830

§ 371 Date: Apr. 21, 2010

(30) Foreign Application Priority Data
Jun. 23, 2005 (KR) 10-2005-0054371
Mar. 30, 2006 (KR) 10-2006-0029071

(51) Int. Cl. C12Q 1/68; G01N 33/53; C12N 5/02; G01N 33/566; C40B 30/04; C40B 40/06; C40B 40/10; C07K 14/435; C07K 16/18; C07H 21/04

(52) U.S. Cl. 435/6; 435/7.92; 435/375; 436/501; 506/9; 506/16; 506/18; 530/350; 530/387.9; 536/23.1; 536/24.31; 536/24.33

(57) ABSTRACT

A genetic polymorphism associated with myocardial infarction is provided. More particularly, provided are a polynucleotide including a single nucleotide polymorphism (SNP) or a haplotype associated with myocardial infarction, a polynucleotide hybridized with the polynucleotide, a polypeptide encoded by one of the polynucleotides, an antibody bound to the polypeptide, a microarray and a kit including one of the polynucleotides, a myocardial infarction diagnosis method, a SNP detecting method and a method of screening pharmaceutical compositions for myocardial infarction.

### CROSS-REFERENCE TO RELATED PATENT APPLICATION

This application claims the benefit of Korean Patent Application No. 10-2005-0054371 and 10-2006-0029071, filed on Jun. 23, 2005 and 30 Mar. 2006, in the Korean Intellectual Property Office, the disclosure of which is incorporated herein in its entirety by reference.

### TECHNICAL FIELD

The present invention relates to a genetic polymorphism associated with myocardial infarction and the uses thereof.

### BACKGROUND ART

99.9% of base sequences of the human genome are identical. Diversity in individuals' appearance, behavior and susceptibility to certain diseases is caused by partial differences in the remaining 0.1% of the base sequences in the human genome. That is, differences in about 3 million base sequences of the human genome account for the diversity among individuals, communities, races and peoples. The differences in base sequences contribute to the differences in disease distributions as well as phenotypic distinctions such as skin color of different races. There are about 3 billion base pairs, and single base pairs that vary from person to person are positioned at intervals of about 1.0 kb. There are about 3 million base pairs in total that vary from person to person, and variations in these base pairs are referred to as Single Nucleotide Polymorphisms (SNPs). Causes of the diversity among individuals and communities or the difference between a disease group and a normal group may be found through the analysis of the 3 million SNPs, and analysis of entire base sequences is unnecessary.

The prime object of genetics is to map phenotypic differences such as diseases in humans to variations in DNA. A polymorphic marker existing in all genomes is the best means of obtaining this object. Microsatellite markers have been commonly used as polymorphic markers to distinguish individuals and find genes related to genetic diseases having, but SNPs have drawn attention with the development of DNA chips. Automatic SNP detection on a large scale is possible because of the high frequency of SNPs, the safety of using SNPs, and the even distribution of SNPs across all genomes. SNPs will contribute to predictive medical science, which is a new branch of medical science. For example a revolutionary process of predicting diseases of individuals and investigating individual's reactions to a certain medical supply can be performed by implementing up-to-date biotechnology such as a DNA chip technique and a high speed DNA sequence analysis technique.

The study of SNPs involves an analysis of genotypes evenly distributed throughout the population. By studying SNPs, a population may be divided according to genotype, and if a disease group is significantly distributed according to a genotype, the relationship between the genotype and the disease can be established. In most studies of SNPs, if a single genotype or several genotypes have significantly different distributions in a disease group and a normal group, the differences in disease frequency according to the genotype may be analyzed.

About 510,000 SNPs, approximately one sixth of the 3 million SNPs in human genomes, exist in genes. It is very important to know the distribution of such SNPs in genes because the SNPs are directly related to gene expression or protein functions. If a genotype associated with a certain disease can effect a change in a gene expression or a protein function, the gene or the protein is likely to be a cause of the disease. In this case, the gene can be the target gene for disease detection and treatment. The susceptibility to the disease may also be analyzed using SNP analysis of the gene.

SNPs in transcription regulatory regions of a gene sequence, such as a promoter, can regulate the quantity of expressed genes. On rare occasions, SNPs also influence the stability and translation efficiency of RNA located in sequences at exon-intron boundaries affecting RNA splicing or in a 3′-untranslated region (3′-UTR). That is, the SNPs that are located in an encoding region or a transcription and translation regulatory region may be a useful index for determining susceptibility to a disease. Although some SNPs are located in an encoding region or a transcription and translation regulatory region, other SNPs which are not expected to affect protein expression and/or function could be found associated with a disease. International researches are being tried to reveal the role of such SNPs in human diseases.

Genotype analysis using haplotypes provides more accurate results since haplotypes have more information than SNPs and contain information about linkage disequilibrium. Particularly, when several SNPs are densely distributed in a gene, the gene analysis using haplotypes, which have combined information regarding adjacent SNPs, is effective for finding the relation between a gene function and a disease. For example, a haplotype of Five Lipo-oxygenase Activation Pepetide (FLAP) has been found to be a more accurate marker for a heart attack than a SNP, and has been reported to be a critical cause of heart attacks (Helgadotiir et al., Nat Genet. 2004 March; 36(3):233-9). Since then, Decode Genetics has performed research on an inhibitor of the enzyme containing FLAP haplotypes for preventative medicine for heart attacks based on the reported results of the haplotype, and a second phase clinical trial has already commenced.

Meanwhile, cardiovascular disease is a major cause of death in industrialized countries around the world, and has been a major cause of death in the Republic of Korea since the 1970s. According to the Korean National Statistical Office, in 2003, 22,000 out of 246,000 deaths (9087 per 100,000, or 9.1%) were the result of cardiac disorders and hyperpiesia, which are the third leading cause of death in Korea following cancer and cerebrovascular disease.

Coronary artery disease, which ranks high among cardiovascular disease, is usually caused by arteriosclerosis, the blocking or narrowing of coronary arteries supplying blood to the heart. Blocking of the coronary artery indicates myocardial infarction and narrowing of the coronary artery indicates angina pectoris. The causes of coronary artery disease are known to be hyperlipidemia (hypercholesterolemia), hyperpiesia, smoking, diabetes, genetic inheritance, obesity, lack of exercise, stress and menopause. A subject having complex factors of coronary artery disease has a higher risk of incidence.

### DISCLOSURE OF INVENTION

### Technical Problem

Currently, X-ray and ultrasonography of the interior of the heart and coronary artery can be used for cardiovascular disease diagnosis. However, as with the diagnosis or prognosis of other various cardiovascular and complicated diseases including myocardial infarction 7using other physical techniques, the diagnosis or prediction can be performed only when the diseases are at an advanced stage.

### Technical Solution

The present inventors found SNPs and haplotypes as a result of research to find SNPs associated with myocardial infarction, which makes it possible to predict the incidence probability of and genetic susceptibility to myocardial infarction.

### SUMMARY OF THE INVENTION

The present invention provides a polynucleotide containing a single nucleotide polymorphism (SNP) associated with myocardial infarction.

The present invention also provides a polynucleotide containing a haplotype associated with myocardial infarction.

The present invention also provides a polynucleotide specifically hybridized with the polynucleotide.

The present invention also provides a polypeptide encoded by the polynucleotide.

The present invention also provides an antibody specifically bound to the polypeptide.

The present invention also provides a microarray for detecting SNPs including the polynucleotide.

The present invention also provides a kit for detecting SNPs including the polynucleotide.

The present invention also provides a method of identifying a subject having a changed risk of incidence of myocardial infarction.

The present invention also provides a method of detecting SNPs or haplotypes in nucleic acid molecules.

The present invention also provides a method of screening pharmaceutical compositions for myocardial infarction.

The present invention also provides a method of regulating gene expression.

According to an aspect of the present invention, there is provided a polynucleotide of nucleotide sequences of SEQ ID NOS: 1 to 7, 9, 10 and 14 including at least 8 contiguous nucleotides and the 101^{st} base of the nucleotide sequence and complementary polynucleotides of the nucleotide sequences.

According to another aspect of the present invention, there is provided a polynucleotide including nucleotide sequences of SEQ ID NOS: 1 to 14, wherein each nucleotide sequence includes at least 8 contiguous nucleotides and the 101^{st} base of the nucleotide sequence or a complementary polynucleotide of the nucleotide sequences.

According to another aspect of the present invention, there is provided a polynucleotide specifically hybridized with the polynucleotide.

According to another aspect of the present invention, there is provided a polypeptide encoded by the polynucleotide.

According to another aspect of the present invention, there is provided an antibody specifically bound to the polypeptide.

According to another aspect of the present invention, there is provided a microarray for detecting SNPs including the polynucleotide, the polypeptide encoded by the polynucleotide or cDNA thereof.

According to another aspect of the present invention, there is provided a kit for detecting SNPs including the polynucleotide, the polypeptide encoded by the polynucleotide or cDNA thereof.

According to another aspect of the present invention, there is provided a method of identifying a subject having a changed risk of incidence of myocardial infarction including isolating a nucleic acid sample from the subject and determining an allele at a polymorphic site of one or more polynucleotides among SEQ ID NOS: 1 to 7, 9, 10 and 14 or a polynucleotide including SEQ ID NOS: 1 to 14, wherein the polymorphic site is positioned at the 101^{st} nucleotide of the nucleotide sequences.

According to another aspect of the present invention, there is provided a method of detecting SNPs or haplotypes in nucleic acid molecules including contacting a test sample having nucleic acid molecules with a reagent specifically hybridized under strict conditions with a polynucleotide of nucleotide sequences of SEQ ID NOS: 1 to 7, 9, 10 and 14 or a polynucleotide including SEQ ID NOS: 1 to 14, the nucleotide sequence containing at least 8 contiguous nucleotides and the 101^{st} base of the nucleotide sequence or a complementary polynucleotide of the nucleotide sequences and detecting the formation of a hybridized double-strand.

According to another aspect of the present invention, there is provided a method of screening pharmaceutical compositions for myocardial infarction including contacting a candidate material with a polypeptide encoded by a polynucleotide of nucleotide sequences of SEQ ID NOS: 1 to 7, 9, 10 and 14 or a polynucleotide including SEQ ID NOS: 1 to 14, the nucleotide sequence containing at least 8 contiguous nucleotides and the 101^{st} base of nucleotide sequence or a complementary polynucleotide of the nucleotide sequences under proper conditions for the formation of a binding complex and detecting the formation of the binding complex from the polypeptide and the candidate material.

According to another aspect of the present invention, there is provided a method of regulating gene expression including binding an anti-sense nucleotide or Si RNA with a polynucleotide of nucleotide sequences of SEQ ID NOS: 1 to 7, 9, 10 and 14 or a polynucleotide including SEQ ID NOS: 1 to 14, the nucleotide sequence containing at least 8 contiguous nucleotides and the 101^{st} base of the nucleotide sequence or a complementary polynucleotide of the nucleotide sequence, wherein the anti-sense nucleotide or Si RNA is specific to the polynucleotide.

The above aspects and advantages of the present invention will become more apparent by describing in detail exemplary embodiments thereof.

### DETAILED DESCRIPTION OF THE INVENTION

A single nucleotide polymorphism (SNP) associated with myocardial infarction according to an embodiment of the present invention includes a polynucleotide of nucleotide sequences of SEQ ID NOS: 1 to 7, 9, 10 and 14 containing at least 8 contiguous nucleotides and the 101^{st} base of the nucleotide sequence, and complementary polynucleotides of the nucleotide sequences.

A polynucleotide containing one of the nucleotide sequences of SEQ ID NOS: 1 to 7, 9, 10 and 14 is a polymorphic sequence. A polymorphic sequence is a nucleotide sequence including polymorphic sites where SNPs exist in a polynucleotide sequence. The polynucleotide may be DNA or RNA.

Herein, SNPs may be the most commonly found single base-pair variation among DNA sequence polymorphisms shown in about every 1 kb in the DNA of individuals.

SNPs according to an embodiment of the present invention exist within NFkB1, which is a major transcription regulating gene in an inflammation reaction. It has not been reported that genetic variations of inflammation related genes are highly associated with cardiovascular disease (Auer et al., Am J. Pharmacogenomics. 2003; 3(5):317-28). Specifically it has not been reported that the SNPs in the NFkB1 gene are associated with cardiovascular disease.

In Examples of the present invention, a series of selections from NFkB1 gene was made in order to find SNPs closely associated with myocardial infarction. DNA was isolated from blood of myocardial infarction patients and normal persons and amplified. After analyzing the SNP sequence in the NFkB1 gene of the DNA, SNPs and the genotypes thereof having very different appearance frequencies in the patients and normal persons were identified. 10 SNPs and the genotypes thereof which were identified in Examples of the present invention are listed in Table 1. In the meantime, the appearance frequencies of the SNPs of SEQ ID NOS: 8 and 12 in the patients having myocardial infarction were remarkably different from the appearance frequencies of the SNPs of SEQ ID NOS: 8 and 12 in normal persons, which has been disclosed in the Korean Patent Application No. 2005-47195. The appearance frequencies of the SNPs of SEQ ID NOS: 11 and 13 in the patients having myocardial infarction were not different from the appearance frequencies of the SNPs of SEQ ID NOS: 13 and 14 in normal persons.

TABLE 1

| | | SEQ ID | | | SNP | p | | | |
|---|---|---|---|---|---|---|---|---|---|
| No | alias_id | NO: | A1 | A2 | function | value | OR | OR_LB | OR_UB |
| 1 | MI_2013 | 1 | T | C | promoter | 0.0041 | 1.50 | 1.13 | 1.99 |
| 2 | MI_2606 | 2 | A | G | 5′ | 0.0013 | 1.59 | 1.21 | 2.10 |
| | | | | | UTR | | | | |
| 3 | MI_2607 | 3 | T | C | 5′ | 0.0134 | 1.44 | 1.09 | 1.90 |
| | | | | | UTR | | | | |
| 4 | MI_2608 | 4 | T | C | 5′ | 0.003 | 1.63 | 1.22 | 2.18 |
| | | | | | UTR | | | | |
| 5 | MI_2612 | 5 | T | C | intron 4 | 0.0205 | 1.35 | 1.01 | 1.79 |
| 6 | MI_2614 | 6 | T | C | intron 5 | 0.0048 | 1.54 | 1.15 | 2.05 |
| 7 | MI_2615 | 7 | A | G | intron 5 | 0.0035 | 1.55 | 1.16 | 2.07 |
| 8 | MI_1329 | 8 | C | G | intron 8 | 0.0025 | 1.56 | 2.08 | 1.18 |
| 9 | MI_2620 | 9 | T | C | intron | 0.0067 | 1.53 | 1.14 | 2.04 |
| | | | | | 16 | | | | |
| 10 | MI_2621 | 10 | A | G | intron | 0.0009 | 1.52 | 1.15 | 2.00 |
| | | | | | 17 | | | | |
| 11 | MI_2022 | 11 | T | A | intron | 0.3098 | 1.37 | 2.33 | 0.81 |
| | | | | | 22 | | | | |
| 12 | MI_1377 | 12 | C | T | intron | 0.0181 | 1.47 | 2.00 | 1.10 |
| | | | | | 23 | | | | |
| 13 | MI_2026 | 13 | T | C | 3′ | 0.1526 | 1.52 | 2.50 | 0.93 |
| | | | | | UTR | | | | |
| 14 | MI_2027 | 14 | T | C | 3′ | 0.0152 | 1.53 | 1.14 | 2.04 |
| | | | | | UTR | | | | |

In table 1, ‘alias_id’ is a SNP number arbitrarily designated by the inventors of the present invention.

‘SEQ ID NO:’ is the polynucleotide sequence identification number including the SNP enclosed with the specification of the present invention.

A1 (allele ‘1’) and A2 (allele ‘2’) respectively represent a low mass allele and a high mass allele in sequencing experiments according to a homogeneous MassEXTEND™ technique of Sequenom, and are arbitrarily designated for convenience of experiments.

‘SNP_function’ is the role performed by the SNP within the gene.

‘P-value’ is the p-value obtained by inspecting the gene using Fisher's exact test. When the p-value was 0.05 or less, it was determined that the genotype between the disease group and the normal group was not identical, i.e. significant.

‘OR’ is the odds ratio indicating the ratio of the probability of the SNP in the disease group to the probability of the SNP in the normal group based on genotype.

‘OR_LB’ and ‘OR_UB’ respectively indicate the lower limit and the upper limit of the 95% confidence interval of the odds ratio. When the odds ratio exceeds 1, allele ‘1’ is the risk factor. When the confidence interval includes 1, it can not be determined that the relation between the genotype and the disease is significant.

The polynucleotide according to an embodiment of the present invention includes certain haplotypes of the NFkB1 gene. The polynucleotide for diagnosis of myocardial infarction according to an embodiment of the present invention may include nucleotide sequences of SEQ ID NOS: 1 to 14.

The haplotype of the present embodiment indicates a group of alleles closely connected to each other, and not separated during the gene recombination process.

TABLE 2

| | | Hap. Freq | | | | |
|---|---|---|---|---|---|---|
| ID | haplotype | total_freq | y.0 con_freq | y.1 cas_freq | p. val | Hap. Score |
| 1 | 22211221121112 | 0.04258 | 0.07368 | 0.01584 | 0.00012 | −3.84477 |
| 2 | 11112221111112 | 0.48146 | 0.54199 | 0.42976 | 0.00118 | −3.24363 |
| 3 | 11111112221211 | 0.00615 | 0.01344 | NA | 0.01427 | −2.45043 |
| 4 | 12112221122122 | 0.04856 | 0.05789 | 0.04046 | 0.23779 | −1.18053 |
| 5 | 22221112221122 | 0.00582 | 0.00534 | 0.00679 | 0.93281 | 0.0843 |
| 6 | 22221112221111 | 0.00604 | 0.00526 | 0.00679 | 0.78959 | 0.26685 |
| 7 | 11112221111111 | 0.01239 | 0.00797 | 0.01592 | 0.30841 | 1.01856 |
| 8 | 21221112221122 | 0.00852 | NA | 0.01584 | 0.0133 | 2.47563 |
| 9 | 22221112221211 | 0.31498 | 0.26279 | 0.35968 | 0.00327 | 2.94088 |
| 10 | 11112221122122 | 0.02437 | 0.00264 | 0.04299 | 0.00095 | 3.30504 |
| 11 | 11211221121112 | 0.01825 | NA | 0.03394 | 0.00025 | 3.65851 |

In Table 2, ‘haplotype’ indicates the ordered sequence of the genotypes of the SNPs of SEQ ID NOS: 1 to 14, where ‘1’ and ‘2’ respectively represent allele ‘1’ and allele ‘2’. For example, the haplotype No. 2 includes the nucleotide sequences of SEQ ID NOS: 1 to 14 and the genotypes of the SNPs are respectively T, A, T, T, C, C, G, C, T, A, T, C, T and C.

‘Hap.Freq total_freq’ indicates the frequency of the haplotype in the disease group and the normal group.

‘P-value’ indicates the results of statistical analysis of the difference between haplotype frequencies of the disease group and the normal group. The software used in the analysis is ‘haplo.score,’ which is conventional published statistical analysis software.

When ‘Hap.Freq total_freq’ was 0.05 or higher and ‘p-value’ was 0.05 or less, the relation between the haplotype and myocardial infarction was determined to be significant. In this case, an SNP site at the 101^{st} base of a polynucleotide including the nucleotide sequences of SEQ ID NOS: 1 to 14 consisting of the haplotype may be a risk allele. That is, haplotype Nos. 2 and 9 in Table 2 may be used to distinguish between the disease group and the normal group having high frequencies in the population.

In an embodiment of the present invention, a polynucleotide containing a SNP may include at least 8 contiguous nucleotides, for example, 8 to 70 contiguous nucleotides.

In an embodiment of the present invention, a polynucleotide is specifically hybridized with a polynucleotide including the SNP or the haplotype. That is, the polynucleotide is specifically hybridized with a polynucleotide of nucleotide sequences of SEQ ID NOS: 1 to 7, 9, 10 and 14 including at least 8 contiguous nucleotides and the 101^{st} base of the nucleotide sequence or a complementary polynucleotide of the nucleotide sequences; or a polynucleotide including nucleotide sequences of SEQ ID NOS: 1 to 14 in which each nucleotide sequence includes at least 8 contiguous nucleotides and the 101^{st} base of nucleotide sequence or a complementary polynucleotide of the nucleotide sequences. The polynucleotide may be allele-specific.

The polynucleotide may include at least 8 contiguous nucleotides, for example, 8 to 70 contiguous nucleotides.

The allele-specific polynucleotide is specifically hybridized with each allele of the polynucleotide. The hybridization can be performed to distinguish the bases at polymorphic sites of polymorphic sequences of SEQ ID NOS: 1 to 14. The hybridization can be carried out under strict conditions, for example, in a salt concentration of 1 M or less and at a temperature of 25° C. or higher. For example, 5×SSPE (750 mM NaCl, 50 mM Na Phosphate, 5 mM EDTA, pH 7.4) and 25 to 30° C. may be suitable conditions for the allele-specific probe hybridization.

In an embodiment of the present invention, the allele-specific polynucleotide can be a primer. The primer is a single-strand oligonucleotide capable of initiating template-directed DNA synthesis in an appropriate buffer under appropriate conditions, for example, in the presence of four different nucleotide triphosphates and a polymerizing agent such as DNA, RNA polymerase or reverse transcriptase at a proper temperature. The length of the primer may vary according to the purpose of use, but is 15 to 30 nucleotides in an embodiment of the present invention. A short primer molecule can require a lower temperature to be stably hybridized with the template. The primer sequence does not necessarily need to be completely complementary to the template, but can be sufficiently complementary to be hybridized with the template. The 3′ end of the primer is arranged to correspond to the polymorphic sites of SEQ ID NOS. 1 to 14. The primer is hybridized with the target DNA including the polymorphic site and initiates amplification of an allele having complete homology to the primer. The primer and another primer hybridized with the other allele are used as a primer pair. Amplification is performed from the two primers, indicating that there is a specific allele in the polynucleotide. According to an embodiment of the present invention, the primer includes a polynucleotide fragment used in a ligase chain reaction (LCR). For example, the primer may be a polynucleotide of nucleotide sequences of SEQ ID NOS: 15 to 56 in Table 3.

In an embodiment of the present invention, an allele specific polynucleotide may be a probe. The probe is a hybridization probe, which is an oligonucleotide capable of binding specifically to a complementary strand of a nucleic acid. Such a probe includes a peptide nucleic acid introduced by Nielsen et al., Science 254, 1497-1500 (1991). According to an embodiment of the present invention, the probe is an allele-specific probe. When a polymorphic site is located in nucleic acid fragments derived from two members of the same species, the allele-specific probe can hybridize with the DNA fragment derived from one member but not with the DNA fragment derived from the other member. In this case, the hybridization conditions can be suitable for hybridization with only one allele by facilitating a significant difference in intensities of hybridization for different alleles. According to an embodiment of the present invention, the probe is arranged such that its central site is the polymorphic site of the sequence, for example the 7^{th} position in a probe consisting of 15 nucleotides, or the 8^{th} or 9^{th} position in a probe consisting of 16 nucleotides. In this way, a difference in hybridization for different alleles can be obtained. According to an embodiment of the present invention, the probe can be used in a diagnosis method for detecting an allele, etc. The diagnosis method may be Southern blotting in which detection is performed using the hybridization of nucleic acids, or a method in which a microarray to which the probe is bound in advance is used.

A polypeptide according to an embodiment of the present invention is encoded by a polynucleotide including the SNP or the haplotype. The polypeptide is encoded by a polynucleotide of nucleotide sequences of SEQ ID NOS: 1 to 7, 9, 10 and 14 including at least 8 contiguous nucleotides and 101^{st} base of the nucleotide sequence or a complementary polynucleotide of the nucleotide sequences, or by a polynucleotide including nucleotide sequences of SEQ ID NOS: 1 to 14 in which each nucleotide sequence includes at least 8 contiguous nucleotides and the 101^{st} base of nucleotide sequence or a complementary polynucleotides of the nucleotide sequences.

An antibody according to an embodiment of the present invention specifically binds to the polypeptide. The antibody may be a monoclonal antibody.

A microarray according to an embodiment of the present invention includes a polynucleotide of nucleotide sequences of SEQ ID NOS: 1 to 7, 9, 10 and 14 including at least 8 contiguous nucleotides and the 101^{st} base of the nucleotide sequence or a complementary polynucleotide of the nucleotide sequences, a polynucleotide including nucleotide sequences of SEQ ID NOS: 1 to 14 in which each nucleotide sequence includes at least 8 contiguous nucleotides and the 101^{st} base of nucleotide sequence or a complementary polynucleotide of the nucleotide sequences, a polypeptide encoded by one of the polynucleotides, or cDNA thereof.

The microarray may be prepared using a conventional method known to those skilled in the art using the polynucleotide, a probe or a polynucleotide hybridized with the probe, the polypeptide encoded by one of the polynucleotides or cDNA thereof.

For example, the polynucleotide may be fixed to a substrate coated with an active group of amino-silane, poly-L-lysine or aldehyde. Also, the substrate may be composed of silicon, glass, quartz, metal or plastic. A polynucleotide may be fixed to the substrate by micropipetting using a piezoelectric method or by using a spotter in the shape of a pin.

A kit according to an embodiment of the present invention includes a polynucleotide of nucleotide sequences of SEQ ID NOS: 1 to 7, 9, 10 and 14 including at least 8 contiguous nucleotides and 101^{st} base of the nucleotide sequence and complementary polynucleotides of the nucleotide sequences, a polynucleotide including nucleotide sequences of SEQ ID NOS: 1 to 14 in which each nucleotide sequence includes at least 8 contiguous nucleotides and the 101^{st} base of nucleotide sequence or a complementary polynucleotide of the nucleotide sequences, a polypeptide encoded by one of the polynucleotides, or cDNA thereof.

The kit may further include a primer set used for isolating DNA including SNPs from diagnosed subjects and amplifying the DNA. The appropriate primer set may be determined by those skilled in the art. For example, the primer set in Table 3 may be used. Also, the kit may further include a reagent for a polymerizing reaction, for example dNTP, various polymerases and colorants.

An identifying method according to an embodiment of the present invention includes using the SNP or the haplotype to identify a subject having a changed risk of incidence of myocardial infarction.

The identifying method includes isolating a nucleic acid sample from a subject and determining an allele at polymorphic sites of one or more polynucleotides among nucleotide sequences of SEQ ID NOS: 1 to 7, 9, 10 and 14 or a polynucleotide including nucleotide sequences of SEQ ID NOS: 1 to 14, wherein the polymorphic site is positioned at the 101^{st} nucleotide of the nucleotide sequences.

In an embodiment of the present invention, the isolation of the DNA from the subject can be carried out by performing a method known to those skilled in the art. For example, DNA can be directly purified from tissues or cells or a specific region can be amplified using a polymerase chain reaction (PCR), etc. and isolated. In the detailed description, DNA refers not only to DNA, but also to cDNA synthesized from mRNA. Nucleic acids can be obtained from a subject using PCR amplification, ligase chain reaction (LCR) (Wu and Wallace, Genomics 4, 560 (1989), Landegren, etc., Science 241, 1077 (1988)), transcription amplification (Kwoh, etc., Proc. Natl. Acad. Sci. USA 86, 1173 (1989)), self-sustained sequence replication (Guatelli, etc., Proc. Natl. Acad. Sci. USA 87, 1874 (1990)) or Nucleic Acid Sequence Based Amplification (NASBA).

The isolated DNA may be sequenced through various methods known to those skilled in the art. For example, the nucleotides of nucleic acids may be directly sequenced using a dideoxy method. Also, the nucleotides of the polymorphic sites may be sequenced by hybridizing the DNA with a probe containing the sequence containing the SNP site and a complementary probe thereof, and examining the degree of the hybridization. The degree of hybridization may be measured using a method of indicating a detectable index of the target DNA and specifically detecting the hybridized target, or using an electrical signal detecting method.

Particularly, the sequencing may be carried out using allele-specific probe hybridization, allele-specific amplification, sequencing, 5′ nuclease digestion, molecular beacon assay, oligonucleotide ligation assay, size analysis or single-stranded conformation polymorphism method.

In an embodiment of the present invention, the method of diagnosing myocardial infarction may further include judging that the subject has an increased risk of incidence of myocardial infarction when an allele at a polymorphic site of one or more polynucleotides of nucleotide sequences of SEQ ID NOS: 1 to 7, 9, 10 and 14 or a polynucleotide including nucleotide sequences of SEQ ID NOS: 1 to 14 is a risk allele.

According to an embodiment of the present invention, the risk allele is determined based on the allele ‘1’. When the frequency of the allele ‘1’ in the disease group is higher than that in the normal group, allele ‘1’ is regarded as a risk allele. In the opposite case, allele ‘2’ is regarded as a risk allele. Subjects having more risk alleles have a higher probability of having myocardial infarction.

In an embodiment of the present invention, the changed risk may be an increased risk or decreased risk. When the frequency of an allele is higher in the normal group than in the disease group, the changed risk may be a decreased risk. On the other hand, when the frequency of an allele of the SNP is higher in the disease group than in the normal group, the changed risk can be an increased risk.

A method of detecting SNPs or haplotypes in nucleic acid molecules according to an embodiment of the present invention includes contacting a test sample containing nucleic acid molecules with a reagent specifically hybridized under strict conditions with a polynucleotide of a nucleotide sequence of SEQ ID NOS: 1 to 7, 9, 10 and 14 or a polynucleotide including nucleotide sequences of SEQ ID NOS: 1 to 14 in which the nucleotide sequence contains at least 8 contiguous nucleotides and the 101^{st} base of the nucleotide sequence or a complementary polynucleotide of the nucleotide sequences, and detecting the formation of a hybridized double-strand.

The detecting of the formation of a hybridized double-strand is carried out using allele-specific probe hybridization, allele-specific amplification, sequencing, 5′ nuclease digestion, molecular beacon assay, oligonucleotide ligation assay, size analysis or single-stranded conformation polymorphism.

A method of screening pharmaceutical compositions for myocardial infarction according to an embodiment of the present invention includes contacting a candidate material with a polypeptide encoded by a polynucleotide of a nucleotide sequence of SEQ ID NOS: 1 to 7, 9, 10 and 14 or a polynucleotide including nucleotide sequences of SEQ BD NOS: 1 to 14 in which the nucleotide sequence includes at least 8 contiguous nucleotides and the 101^{st} base of the nucleotide sequence or a complementary polynucleotide of the nucleotide sequences under proper conditions for the formation of a binding complex and detecting the formation of the binding complex from the polypeptide and the candidate material.

The detecting the formation of the binding complex may be carried out through coimmunoprecipitation, Radioimmunoassay (RIA), Enzyme Linked ImmunoSorbent Assay (ELISA), Immunohistochemistry, Western Blotting or Fluorescence Activated Cell Sorer (FACS).

A method of regulating gene expression according to an embodiment of the present invention includes binding an anti-sense nucleotide or Si RNA with a polynucleotide of a nucleotide sequence of SEQ ID NOS: 1 to 7, 9, 10 and 14 or a polynucleotide including nucleotide sequences of SEQ ID NOS: 1 to 14 in which the nucleotide sequence includes at least 8 contiguous nucleotides and the 101^{st} base of the nucleotide sequence or a complementary polynucleotides of the nucleotide sequences, wherein the anti-sense nucleotide and Si RNA are specific to the polynucleotide.

### Advantageous Effects

The SNP and haplotype associated with myocardial infarction according to the present invention may be used for diagnosis and treatment of myocardial infarction and gene fingerprint analysis. By using the microarray and kit including the SNP of the present invention, myocardial infarction can be effectively diagnosed. According to the method of analyzing SNPs associated with myocardial infarction of the present invention, the presence or risk of myocardial infarction can effectively be diagnosed.

### BEST MODE

The present invention will now be described in greater detail with reference to the following examples. The following examples are for illustrative purposes only and are not intended to limit the scope of the invention.

### EXAMPLE 1

### SNP Selection

DNA was isolated from blood of a disease group diagnosed with a cardiovascular disease and treated, and DNA was isolated from a normal group not having symptoms of cardiovascular disease, and then an appearance frequency of a specific SNP was analyzed. Both group consisted of Koreans. The SNP of the Examples of the present invention was selected from either a published database (NCBI dbSNP:http://www.ncbi.nlm.nih.gov/SNP/) or a Sequenom website (http://www.realsnp.com/). The SNPs were analyzed using a primer close to the selected SNP.

1-1. Preparation of DNA Sample

DNA was extracted from blood of a disease group consisting of 221 Korean male patients diagnosed with myocardial infarction and treated and DNA was extracted from a normal group consisting of 192 Korean men not having myocardial infarction symptoms. The chromosome DNA extraction was carried out according to a known method (Molecular cloning: A Laboratory Manual, p 392, Sambrook, Fritsch and Maniatis, 2nd edition, Cold Spring Harbor Press, 1989) and the guidelines of a commercially available kit (Gentra system, D-50K). Only DNA having a UV absorvance ratio (260/280 nm) of at least 1.7 was selected from the extracted DNA and used.

1-2. Amplification of the Target DNA

Target DNA containing a certain DNA region including SNPs to be analyzed was amplified using a PCR. The PCR was performed using a conventional method and the conditions were as indicated below. 2.5 ng/ml of target genome DNA was first prepared. Then the following PCR reaction solution was prepared.

Water (HPLC grade) 3.14 μl

10×buffer 0.5 μl

MgCl₂ 25 mM 0.2 μl

dNTP mix (GIBCO)(25 mM/each) 0.04 μl

Taq pol (HotStart)(5 U/μl) 0.02 μl

Forward/reverse primer mix (10 μM) 0.1 μl

DNA 1.00 μl

Total volume 5.00 μl

The forward and reverse primers were selected upstream and downstream from the SNP at proper positions. The primer set is listed in Table 3.

Thermal cycling of a PCR was performed by maintaining the temperature at 95° C. for 15 minutes, cycling the temperature from 95° C. for 30 seconds, to 56° C. for 30 seconds, to 72° C. for 1 minute a total of 45 times, maintaining the temperature at 72° C. for 3 minutes, and then storing at 4° C. As a result, target DNA fragments having 200 nucleotides or less were obtained.

1-3. Analysis of SNP of the Amplified Target DNA

Analysis of the SNPs in the target DNA fragments was performed using a homogeneous Mass Extend (hME) technique from Sequenom. The principle of the hME technique is as follows. First, a primer, also called an extension primer, complementary to bases up to just before the SNP of the target DNA fragment was prepared. Next, the primer was hybridized with the target DNA fragment and DNA polymerization was facilitated. At this time, a reagent (Termination mix, e.g. ddTTP) for terminating the polymerization was added to the reaction solution after the base complementary was added to a first allele base (e.g. ‘A’ allele) among the subject SNP alleles. As a result, when the target DNA fragment included the first allele (e.g. ‘A’ allele), a product having only one base complementary to the added first allele (e.g. ‘T’) was obtained. On the other hand, when the target DNA fragment included a second allele (e.g. ‘G’ allele), a product having a base complementary to the second allele (e.g. ‘C’) and extending to the first allele base (e.g. ‘A’) was obtained. The length of the product extending from the primer was determined using mass analysis to determine the type of allele in the target DNA. Specific experimental conditions were as follows.

First, free dNTPs were removed from the PCR product. To this end, 1.53 μl of pure water, 0.17 μl of a hME buffer and 0.30 μl of shrimp alkaline phosphatase (SAP) were added to a 1.5 ml tube and mixed to prepare a SAP enzyme solution. The tube was centrifuged at 5,000 rpm for 10 seconds. Then, the PCR product was put into the SAP solution tube, sealed, maintained at 37° C. for 20 minutes and at 85° C. for 5 minutes, and then stored at 4° C.

Next, homogeneous extension was performed using the target DNA product as a template. The reaction solution was as follows.

Water (nanopure grade) 1.728 μl

hME extension mix (10× buffer containing 2.25 mM d/ddNTPs) 0.200 μl

Extension primer (each 100 μM) 0.054 μl

Thermosequenase (32 U/μl) 0.018 μl

Total volume 2.00 μl

The reaction solution was mixed well and spin down centrifuged. A tube or plate containing the reaction solution was sealed, maintained at 94° C. for 2 minutes, cycled from 94° C. for 5 seconds, to 52° C. for 5 seconds, to 72° C. for 5 seconds a total of 40 times, and then stored at 4° C. The obtained homogeneous extension product was washed with the resins to reduce the amount of salts (SpectroCLEAN, Sequenom, #10053). The extension primers used for homogeneous extension are indicated in Table 3.

TABLE 3

| Nucleotide | Primer for target DNA | | |
|---|---|---|---|
| containing SNP | amplification (SEQ ID NO:) | | Extension primer |
| (SEQ ID NO:) | Forward primer | Reverse primer | (SEQ ID NO:) |
| 1 | 15 | 16 | 17 |
| 2 | 18 | 19 | 20 |
| 3 | 21 | 22 | 23 |
| 4 | 24 | 25 | 26 |
| 5 | 27 | 28 | 29 |
| 6 | 30 | 31 | 32 |
| 7 | 33 | 34 | 35 |
| 8 | 36 | 37 | 38 |
| 9 | 39 | 40 | 41 |
| 10 | 42 | 43 | 44 |
| 11 | 45 | 46 | 47 |
| 12 | 48 | 49 | 50 |
| 13 | 51 | 52 | 53 |
| 14 | 54 | 55 | 56 |

Mass analysis was performed on the obtained extension product to determine the sequence of a polymorphic site using Matrix Assisted Laser Desorption and IonizationTime of Flight (MALDI-TOF). In the MALDI-TOF, a material to be analyzed was exposed to laser beam, and flew with an ionized matrix (e.g. 3-Hydroxypicolinic acid) in a vacuum to a detector. The flying time to the detector was calculated to determine the mass. A light material could reach the detector in a shorter amount of time than a heavy material. The nucleotide sequences of SNPs in the target DNA may be determined based on differences in mass and known nucleotide sequences of the SNPs.

1-4, Selection of SNP

Allele frequencies in the disease group consisting of 221 Korean male patients diagnosed with myocardial infarction and treated and allele frequencies of the normal group consisting of 192 Korean men not having symptoms of myocardial infarction were compared. The Fisher's exact test was performed based on the frequency as an association test. In the allele association test of the SNP, when the p-value was 0.05 or less, the SNP was regarded as a noticeable genetic marker.

The effect size was assumed using an allele odds ratio at a 95% confidence interval. When the normal group had a higher frequency of a given allele than the disease group, the allele was determined to be associated with a decreased risk of myocardial infarction and the other allele was determined to be the risk allele of myocardial infarction. On the other hand, when the disease group had a higher frequency of a given allele than the normal group, the allele was determined to be the risk allele.

The results are listed in Table 1. As indicated in Table 1, 10 SNPs associated with myocardial infarction were newly identified in NFkB1 gene.

1-5. Selection of Haplotype

The statistical significance of a haplotype including 14 SNPs of SEQ ID NOS: 1 to 14 was investigated using 221 male patients diagnosed with myocardial infarction and treated and 191 men not having symptoms of myocardial infarction. The results are listed in Table 2. As shown in Table 2, several haplotypes that had a p-value of 0.05 or less and showed a significant difference in frequencies in the disease group and the patient group were found. Among them, haplotype Nos. 2 and 9 were determined to be major haplotypes due to their relatively high frequencies.

### EXAMPLE 2

### Preparation of SNP Immobilized Microarray

A microarray was prepared by immobilizing the selected SNPs on a substrate. That is, polynucleotides of nucleotide sequences of SEQ ID NOS: 1 to 7, 9, 10 and 14 including 20 contiguous nucleotides and were immobilized on the substrate, wherein each SNP (101^{st} base of the nucleotide sequence) was located at the 11^{th} of the 20 nucleotides.

First, a N-terminal end of each of the polynucleotides were substituted with an amine group and the polynucleotides were spotted onto a silylated slide (Telechem) where 2×SSC (pH 7.0) of a spotting buffer was used. After spotting, binding was induced in a drying machine and free oligonucleotides were removed by washing with a 0.2% SDS solution for 2 minutes and with triple distilled water for 2 minutes. The microarray was prepared using denaturation induced by increasing the temperature of the slide to 95° C. for 2 minutes, washing with a blocking solution (1.0 g NaBH₄, PBS (pH 7.4) 300 mL, EtOH 100 mL) for 15 minutes, a 0.2% SDS solution for 1 minute and triple distilled water for 2 minutes, and then drying at room temperature.)

### EXAMPLE 3

### Diagnosis of Myocardiar Infarction using the Microarray

A target DNA was isolated from blood of a subject to diagnose the incidence or possibility of myocardial infarction and was labeled with a fluorescent material using the methods described in Examples 1-1 and 1-2. The fluorescent labeled target DNA was hybridized with the microarray prepared in Example 2 at 42° C. for 4 hours in a UniHyb hybridization solution (TeleChem). The slide was washed twice with 2×SSC at room temperature for 5 minutes and dried in air. The dried slide was scanned using a ScanArray 5000 (GSI Lumonics). The scanned results were analyzed using a QuantArray (GSI Lumonics) and an ImaGene software (BioDiscover). The probability of incidence of myocardial infarction and the susceptibility thereto were measured by identifying whether the subject had the SNP according to an embodiment of the present invention.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A polynucleotide selected from the group consisting of nucleotide sequences of SEQ ID NOS: 1 to 7, 9, 10 and 14 comprising at least 8 contiguous nucleotides and the 101^{st} base of the nucleotide sequence and complementary polynucleotides of the nucleotide sequences.

2. A polynucleotide comprising nucleotide sequences of SEQ ID NOS: 1 to 14, wherein each nucleotide sequence comprises at least 8 contiguous nucleotides and the 101^{st} base of the nucleotide or a complementary polynucleotide of the nucleotide sequences.

3. The polynucleotide of claim 1 or 2, comprising 8 to 70 contiguous nucleotides.

4. A polynucleotide specifically hybridized with the polynucleotide of claim 1 or 2.

5. The polynucleotide of claim 4, comprising 8 to 70 contiguous nucleotides.

6. The polynucleotide of claim 4, being an allele specific probe.

7. The polynucleotide of claim 4, being an allele specific primer.

8. A polypeptide encoded by the polynucleotide of claim 1 or 2.

9. An antibody specifically bound to the polypeptide of claim 8.

10. The antibody of claim 9, being a monoclonal antibody.

11. A microarray for detecting a SNP comprising the polynucleotide of claim 1, 2 or 4, a polypeptide encoded by the polynucleotides of claim 1, 2 or 4, or cDNA thereof.

12. A kit for detecting a SNP comprising the polynucleotide of claim 1, 2 or 4, a polypeptide encoded by the polynucleotide of claim 1, 2 or 4, or cDNA thereof.

13. A method of identifying a subject having a changed risk of incidence of myocardial infarction, the method comprising:
isolating a nucleic acid sample from the subject; and
determining an allele at the polymorphic site of one or more polynucleotides selected from the group consisting of nucleotide sequences of SEQ ID NOS: 1 to 7, 9, 10 and 14, or a polynucleotide comprising nucleotide sequences of SEQ ID NOS: 1 to 14, wherein the polymorphic site is positioned at the 101^{st} nucleotide of the nucleotide sequence.

14. The method of claim 13,
wherein the determining the allele is carried out by performing a method selected from the group consisting of allele-specific probe hybridization, allele-specific amplification, sequencing, 5′ nuclease digestion, molecular beacon assay, oligonucleotide ligation assay, size analysis and single-stranded conformation polymorphism.

15. The method of claim 13, wherein the changed risk is an increased risk.

16. The method of claim 13, wherein the changed risk is a decreased risk.

17. The method of claim 13, further comprising:
judging that the subject has an increased risk of incidence of myocardial infarction when an allele at polymorphic site of one or more polynucleotides selected from the group consisting of nucleotide sequences of SEQ ID NOS: 1 to 7, 9, 10 and 14, or of a polynucleotide comprising nucleotide sequences of SEQ ID NOS: 1 to 14 is a risk allele.

18. A method of detecting a SNP or a haplotype in nucleic acid molecules, the method comprising:
contacting a test sample comprising nucleic acid molecules with a reagent specifically hybridized under strict conditions with a polynucleotide selected from the group consisting of nucleotide sequences of SEQ ID NOS: 1 to 7, 9, 10 and 14, or a polynucleotide comprising nucleotides sequences of SEQ ID NOS: 1 to 14, the nucleotide sequence comprising at least 8 contiguous nucleotides and the 101^{st} base of nucleotide sequence or a complementary polynucleotide of the nucleotide sequences; and
detecting the formation of a hybridized double-strand.

19. The method of claim 18,
wherein the detecting the formation of a hybridized double-strand is carried out by performing a method selected from the group consisting of allele-specific probe hybridization, allele-specific amplification, sequencing, 5′ nuclease digestion, molecular beacon assay, oligonucleotide ligation assay, size analysis and single-stranded conformation polymorphism.

20. A method of screening pharmaceutical compositions for myocardial infarction, the method comprising:
contacting a candidate material with a polypeptide encoded by a polynucleotide selected from the group consisting of nucleotide sequences of SEQ ID NOS: 1 to 7, 9, 10 and 14, or a polynucleotide comprising nucleotide sequences of SEQ ID NOS: 1 to 14, the nucleotide sequence comprising at least 8 contiguous nucleotides and the 101^{st} nucleotide of the nucleotide sequence, or a complementary polynucleotide of the nucleotide sequence under proper conditions for the formation of a binding complex; and
detecting the formation of the binding complex from the polypeptide and the candidate material.

21. The method of claim 20, wherein the detecting the formation of the binding complex is carried out by performing a method selected from the group consisting of coimmunoprecipitation, Radioimmunoassay (RIA), Enzyme Linked ImmunoSorbent Assay (ELISA), Immunohistochemistry, Western Blotting and Fluorescence Activated Cell Sorer (FACS).

22. A method of regulating gene expression, the method comprising:
binding an anti-sense nucleotide or Si RNA with a polynucleotide selected from the group consisting of nucleotide sequences of SEQ ID NOS: 1 to 7, 9, 10 and 14, or a polynucleotide comprising nucleotide sequences of SEQ ID NOS: 1 to 14, the nucleotide sequence comprising at least 8 contiguous nucleotides and the 101^{st} base of the nucleotide sequence, or a complementary polynucleotide of the nucleotide sequence, wherein the anti-sense nucleotide or Si RNA is specific to the polynucleotide.
